(19) United States
(12) Patent Application Publication
(10) Pub. No.: US 2007/0156144 A1
(43) Pub. Date: Jul. 5, 2007

(54) Intramedullary nail

(76) Inventors: Dieter Ulrich, Bern (CH); Robert Frigg, Bettlach (CH)

Correspondence Address: JONES DAY, 222 EAST 41ST STREET, NEW YORK, NY, 10017-6702, US

(21) Appl. No.: 11/645,322

(22) Filed: Dec. 21, 2006

Related U.S. Application Data
(63) Continuation of application No. PCT/CH04/00389, filed on Jun. 24, 2004.

(51) Int. Cl. A61F 2/30

(52) U.S. Cl. 606/62

(57) ABSTRACT

An intramedullary nail made of a metal or a metal alloy comprises a longitudinal axis, a connecting part whose cross section has an area F orthogonal to the longitudinal axis, and a shaft part to be inserted into the medulla whose cross section has an area f also orthogonal to the longitudinal axis where f<F. The metal or metal alloy of the shaft part has greater mechanical strength than the metal or the metal alloy of the connecting part.

### CROSS-REFERENCE TO RELATED APPLICATION

This is a continuation of pending International Application No. PCT/CH2004/000389, filed Jun. 24, 2004, the entire contents of which are expressly incorporated herein by reference thereto.

### FIELD OF THE INVENTION

The invention relates to an intramedullary nail made of a metal or metal alloy.

### BACKGROUND OF THE INVENTION

U.S. Pat. No. 4,875,474 A (BORDER) discloses an intramedullary nail whose central section has a lower wall thickness due to machining and therefore also has a lower strength, which is a disadvantage, in comparison with the proximal and distal end portions of the intramedullary nail.

U.S. Pat. No. 6,261,290 B1 (FRIEDL) discloses a hollow intramedullary nail whose distal shaft part has a smaller wall thickness than the proximal connecting part. Therefore, the shaft part is more flexible than the connecting part, but here again there is the disadvantage of the lower strength of the shaft part. This is a disadvantage that is further potentiated by the various transverse bores in the shaft part.

### SUMMARY OF THE INVENTION

The present invention seeks to remedy this situation. The object of the invention is to create an intramedullary nail shaft part that is more flexible in comparison with the connecting part while nevertheless having the greatest possible strength.

The invention achieves this object with an intramedullary nail having a connecting part and a shaft part. The shaft part is intended to be inserted into the intramedullary canal and has greater mechanical strength than the connecting part.

A high mechanical strength is achieved despite the flexibility imparted to the shaft part. Due to this flexibility, the introduction of the intramedullary nail into the intramedullary canal is facilitated and the greater strength of the material reduces the risk of nail breakage.

Another advantage is obtained due to the lower total weight of the nail due to the smaller wall thickness, approx. 30% lower weight versus a nail having a constant wall thickness. Finally, this also yields a more economical manufacturing process for the intramedullary nail because it can be manufactured more rapidly in comparison with the state of the art and no material is lost due to machining by cutting. This is achieved on the one hand due to the fact that it is possible to start with a prefabricated tube and its wall thickness may still be changed while on the other hand the method is faster on the whole than traditional techniques of metal working.

In another embodiment of the intramedullary nail, the tensile strength of the shaft part shows an increasing gradient in the radial direction—from the longitudinal axis or from the wall of the existing cannulation to the surface of the shaft part. This yields the advantage that it is not necessary to insert a mandrel into the cannulation during the cold forming, thus permitting a simpler manufacturing process.

In yet another embodiment, the tensile strength of the shaft part at first shows a declining gradient and then again an increasing gradient in the radial direction—from the outer surface of the shaft to the longitudinal axis or to the wall of the cannulation. In comparison with a method without inserting a mandrel into the cannulation during cold forming, a greater strength of the material can be achieve here on the whole due to the increase in the tensile strength on the inside of the intramedullary nail.

Depending on the embodiment of the intramedullary nail:
the axial length of the connecting part amounts to at most 30%, preferably at most 10% of the total length of the intramedullary nail;
the surface of the shaft has a maximum roughness Rₐ of 1.6 μM, preferably max. 0.8 μm;
the metal or the metal alloy of the shaft part has at least a 5% higher mechanical strength than the metal or the metal alloy of the connecting part;
the metal or metal alloy of the shaft part has a greater strength than the metal or metal alloy of the connecting part;
the metal or metal alloy of the shaft part has a greater bending strength than the metal or metal alloy of the connecting part;
the metal or metal alloy of the shaft part has a greater torsional strength than the metal or metal alloy of the connecting part;
the metal or metal alloy of the shaft part has a greater fatigue strength than the metal or metal alloy of the connecting part.

In another embodiment of the intramedullary nail, the connecting part and the shaft part has the same composition in terms of materials, so the intramedullary nail can be manufactured in one piece.

In yet another embodiment, the higher mechanical strength of the shaft part is created by cold forming of the metal or metal alloy. The advantage here is essentially the simple way of manufacturing the intramedullary nail.

In another embodiment, the outside diameter D_{tube} of the connecting part is larger than the outside diameter D_{shaft} of the shaft part so that only the shaft part need be manufactured by cold forming.

In yet another embodiment, the outside diameter D_{tube} of the connecting part is equal to the outside diameter D_{shaft} of the shaft part. The advantage of this embodiment is the essentially constant mechanical strength of the intramedullary nail over the entire length.

In another embodiment, the intramedullary nail comprises a cannulation that is concentric with the longitudinal axis, preferably in the form of a cylindrical cavity, whereby the wall thickness “W” of the connecting part is preferably greater than the wall thickness “w” of the shaft part. The wall thickness w conforms to the condition 0.60 W<w<0.85 W.

### BRIEF DESCRIPTION OF THE DRAWINGS

The invention and further embodiments of the invention are explained in greater detail below on the basis of the partially schematic diagrams of several exemplary embodiments.

FIG. 1 shows a longitudinal section through an unmachined tube for a first manufacturing variant;

FIG. 2 shows a cross section along line II-II in FIG. 1 and FIG. 3;

FIG. 3 shows a longitudinal section through an inventive intramedullary nail having a tapering distal portion;

FIG. 4 shows a cross section along line III-III in FIG. 3;

FIG. 5 shows a longitudinal section through an unmachined tube for a second manufacturing variant;

FIG. 6 shows a longitudinal section through the partially machined tube according to FIG. 5 having a tapered shaft part;

FIG. 7 shows a longitudinal section through the completely machined tube according to FIG. 6 with a constant outside diameter as the second variant of an inventive intramedullary nail;

FIG. 8 shows a diagram of the mechanical strength of the shaft of an inventive intramedullary nail; and

FIG. 9 shows a diagram of the mechanical strength of the shaft for a variant of an inventive intramedullary nail.

### DETAILED DESCRIPTION OF THE INVENTION

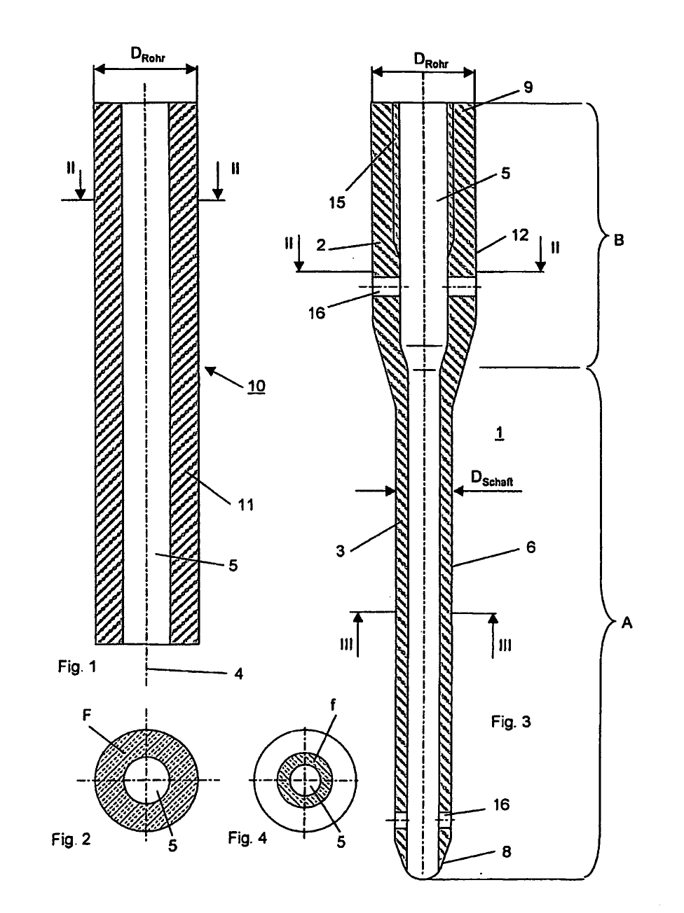

In FIGS. 1 and 5, an unmachined hollow cylindrical or hollow prismatic tube 10 with an outside diameter D_{tube} is shown, serving as the starting piece for the intramedullary nail 1. The tube 10 has a cannulation 5 that is coaxial with the longitudinal axis 4 and is surrounded by the tube wall 11. The cross-sectional area F of the unmachined tube 10 orthogonal to the longitudinal axis 4 is shown in FIG. 2.

FIG. 3 shows the intramedullary nail 1 after cold forming. After cold forming, the intramedullary nail 1 is constricted diametrically on the outside from its distal end 8 on a section A of its length forming the shaft part 3 in comparison with the tube 10 as a starting piece and optionally, depending on the diameter of the mandrel inserted into the cannulation 5 during the shaping, its diameter on the inside is also reduced or unchanged. The surface 6 of the shaft part 3 opens with a conical transition into the surface 12 of the connecting part 2. The cold formed section A of the intramedullary nail 1 has a cross-sectional area f orthogonal to the longitudinal axis 4 (FIG. 4) which is smaller than the cross-sectional area F. Unformed section B of the intramedullary nail 1 adjacent to the proximal end 9 of the intramedullary nail 1 forms the connecting part 2 of the intramedullary nail 1 and has the outside diameter D_{tube} of the unformed tube 10 (FIG. 1). An inside thread 15 is cut into the cannulation 5 in the connecting part 2 from the proximal end. Furthermore, at least one transverse bore 16 with a bore axis running across the longitudinal axis 4 is provided on the connecting part and on the shaft part 3, whereby the angle between the longitudinal axis 4 and the bore axes is typically between 30° and 90°.

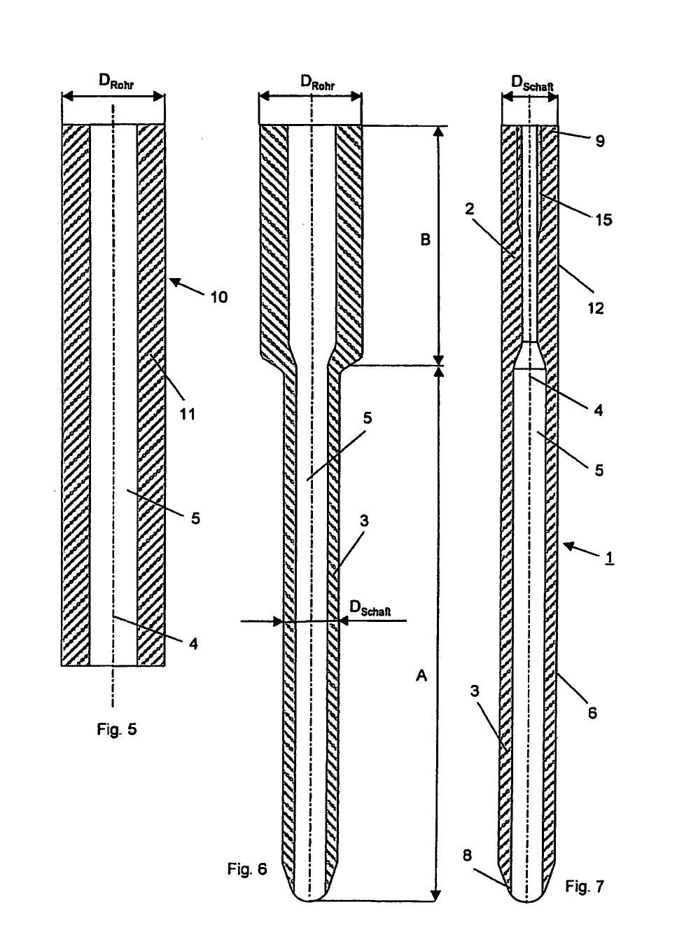

FIG. 6 shows a blank produced from the tube 10 (FIG. 5) and having an outside diameter D_{tube} for another embodiment of the intramedullary nail 1. Here again, the tube 10 (FIG. 5) has been constricted only on the section A with a length of up to an outside diameter D_{shaft}<D_{tube}, as measured from the distal end 8 of the intramedullary nail 1, forming the shaft part 3. The section B of the length of the intramedullary nail 1 as measured from the proximal end 9 is unformed and also has the outside diameter D_{tube}. The cannulation 5 of the blank is optionally either constricted or unchanged in section A, whereby the design of the cannulation 5 after cold forming depends on the diameter of the mandrel inserted into the cannulation 5 during the cold forming process.

FIG. 7 shows the blank depicted in FIG. 6 after a second cold forming which is performed after the shaping of the shaft part 3, which is performed only on section B that forms the connecting part 2. The connecting part 2 (section B) was compressed radially until its outside diameter corresponded to the outside diameter D_{shaft} of the shaft part 3. The cannulation tapers in the transition from the shaft part 3 to the connecting part 2 and has a smaller diameter here in the connecting part 2 than in the shaft part 5. Furthermore, an inside thread 15 is cut in the cannulation 5 in the connecting part 2 from the proximal end 9.

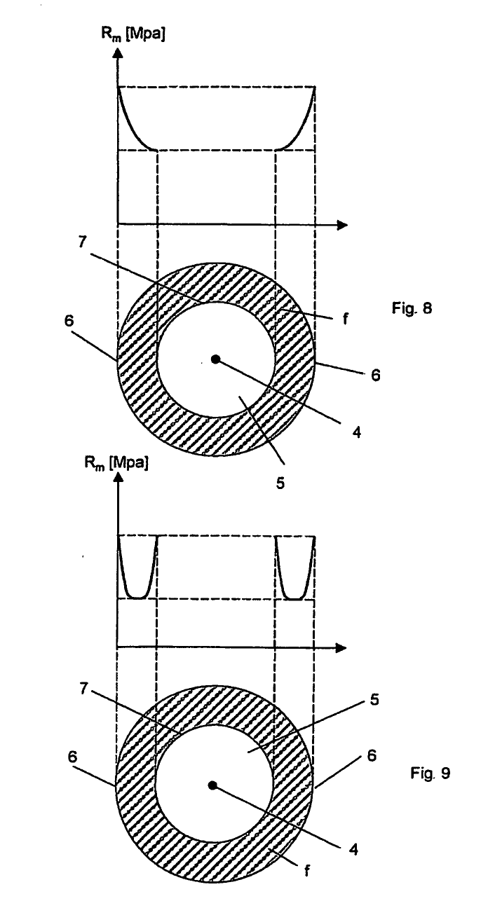

FIG. 8 shows a plot of the tensile strength Rₘ in the tube wall 11 of the cold-formed shaft part 3. The tensile strength Rₘ increases in this case in the radial direction from the wall 7 of the cannulation 5 to the surface 6 of the shaft part 3. Such a plot of the tensile strength Rₘ in the tube wall 11 after cold forming is characteristic of cold forming without the insertion of a mandrel into the cannulation 5.

FIG. 9 shows another plot of the tensile strength Rₘ after the cold forming is concluded. The tensile strength Rₘ in this case has a maximum at the wall 7 of the cannulation 5 and at the surface 6 of the shaft part 3 while a minimum tensile strength Rₘ prevails at the center of the tube wall 11. This plot of the tensile strength Rₘ in the tube wall 11 after cold forming is characteristic of cold forming with insertion of a mandrel into the cannulation 5.

Two different manufacturing methods for the inventive intramedullary nail are given below.

### EXAMPLE 1

The present example corresponds to FIGS. 1 through 4.

A hollow cylindrical or hollow prismatic tube 10 made of stainless steel with a length of typically 100 to 400 mm, an outside diameter of typically 10 to 14 mm and a wall thickness between 1.5 and 4.0 mm is machined over a section A of 70% to 90% of the tube length on the outside by cold forming, section A corresponding approximately to the shaft part 3 of the intramedullary nail 1, so that its outside diameter is reduced to values between 8 and 12 mm and thus the tube 10 is lengthened by 20% to 40%, i.e., is brought to a final length of 120 to 500 mm. By inserting a mandrel with an outside diameter of 5 mm to 10 mm into the cannulation 5 of the tube 10 during the cold forming, the wall thickness of the tube 10 is reduced to 1 to 3 mm.

In comparison with the strength values (Rₘ between 500 and 800 MPa) of the unmachined tube 10, the tube 10 machined according to this invention has 5% to 20% higher strength values (Rₘ values between 600 and 1000 MPa). The blank obtained in this way is processed by applying transverse bores 16 in the shaft part 3 and in the unmachined remainder of the tube, i.e., in the connecting part 2 and a coaxial inside thread 15 in the cannulation 5 in the connecting part 2 to form an intramedullary nail 1.

### EXAMPLE 2

The present example corresponds to FIGS. 5 through 7.

A tube 10 made of stainless steel with a length of typically 100 to 400 mm, an outside diameter of typically 11 to 17 mm and a wall thickness between 1.5 and 4.0 mm is machined by cold forming over a section A of 70% to 90% of the tube length corresponding approximately to the shaft part 3 of the intramedullary nail 1, so that its outside diameter is reduced to values between 11 and 15 mm and thus the tube 10 is lengthened by 20% to 40%, i.e., brought to a final length of 120 to 500 mm.

By inserting a mandrel into the interior of the tube 10, its wall thickness is also reduced to values between 1 and 3 mm. In comparison with the strength values (Rₘ between 500 and 800 MPa) of the unmachined tube 10, the tube 10 machined according to the present invention has 5% to 20% higher strength values (Rₘ values between 600 and 1000 MPa).

In another method step, the mandrel in the interior of the tube 10 is removed and the previously unmachined connecting part 2 of the tube 10 is shaped so that the entire tube 10 has a constant outside diameter. A lower increase in strength occurs in this connecting part because the material can move freely on the inside of the tube. The blank obtained in this way is processed to yield an intramedullary nail 1 by creating transverse bores 16 in the shaft part 3 and the connecting part 2 as well as a coaxial inside thread 15 in the cannulation 5 of the connecting

We claim:

1. An intramedullary nail made of a metal or metal alloy, the nail comprising
a longitudinal axis;
a connecting part having a cross-sectional area F orthogonal to the longitudinal axis; and
a shaft part intended to be inserted into the intramedullary canal and having a cross-sectional area f orthogonal to the longitudinal axis; wherein:
cross-sectional area f is less than cross-sectional area F; and
the shaft part has a greater mechanical strength than the connecting part.

2. The intramedullary nail of claim 1 wherein the tensile strength Rₘ of the shaft part has an increasing gradient in the radial direction from the longitudinal axis to the surface of the shaft part.

3. The intramedullary nail of claim 1 wherein the tensile strength Rₘ of the shaft part has a gradient that first decreases and then increases in the radial direction from the surface of the shaft part toward the longitudinal axis.

4. The intramedullary nail of claim 1 wherein the axial length of the connecting part is at most 30% of the total length of the intramedullary nail.

5. The intramedullary nail of claim 1 wherein the surface of the shaft has a maximum roughness Rₐ of 1.6 μm.

6. The intramedullary nail of claim 1 wherein the metal or metal alloy of the shaft part has a mechanical strength at least 5% greater than that of the metal or metal alloy of the connecting part.

7. The intramedullary nail of claim 1 wherein the metal or metal alloy of the shaft part has a higher tensile strength than the metal or metal alloy of the connecting part.

8. The intramedullary nail of claim 1 wherein the metal or metal alloy of the shaft part has a higher bending strength than the metal or metal alloy of the connecting part.

9. The intramedullary nail of claim 1 wherein the metal or metal alloy of the shaft part has a higher torsional strength than the metal or metal alloy of the connecting part.

10. The intramedullary nail of claim 1 wherein the metal or metal alloy of the shaft part has a higher fatigue strength than the metal or metal alloy of the connecting part.

11. The intramedullary nail of claim 1 wherein the connecting part and the shaft part each have the same composition of material.

12. The intramedullary nail of claim 1 wherein the shaft part is formed by cold forming of the metal or metal alloy.

13. The intramedullary nail of claim 1 wherein the outside diameter D_{tube} of the connecting part is greater than the outside diameter D_{shaft} of the shaft part.

14. The intramedullary nail of claim 1 wherein the outside diameter D_{tube} of the connecting part is the same as the outside diameter D_{shaft} of the shaft part.

15. The intramedullary nail of claim 1 having a cannulation in the form of a cylindrical cavity concentric with the longitudinal axis.

16. The intramedullary nail of claim 15 wherein:
the connecting part has a wall thickness “W;”
the shaft part has a wall thickness “w;” and
wall thickness “W” is greater than wall thickness “w.”

17. The intramedullary nail of claim 16 wherein the wall thickness “w” conforms to the condition 0.60 W<w<0.85 W.

18. A method of manufacturing an intramedullary nail comprising:
cold forming a shaft part of a rod or a tube having a constant cross-sectional area F such that the cross-sectional area F of the shaft part is reduced to f<F.

19. The method of claim 18 wherein the cold forming comprises:
maintaining a constant outside diameter of the tube; and
reducing the wall thickness of the shaft part by cold forming.

20. The method of claim 19 further comprising:
lengthening axially the shaft part; and
reducing the outside diameter of the shaft part.
